(19) United States
(12) Patent Application Publication
(10) Pub. No.: US 2014/0273036 A1
(43) Pub. Date: Sep. 18, 2014

(54) LUMINESCENT DETECTION OF INORGANIC PHOSPHATE AND COUPLED REACTIONS

(71) Applicant: PROMEGA CORPORATION, Madison, WI (US)

(72) Inventors: Subhanjan Mondal, Middleton, WI (US); Kuei-Hsuan Hsiao, Madison, WI (US); Said Goueli, Fitchburg, WI (US)

(73) Assignee: PROMEGA CORPORATION, Madison, WI (US)

(21) Appl. No.: 14/205,909

(22) Filed: Mar. 12, 2014

Related U.S. Application Data
(60) Provisional application No. 61/777,002, filed on Mar. 12, 2013.

(51) Int. Cl. C12Q 1/66

(52) U.S. Cl. CPC C12Q 1/66
USPC 435/8

(57) ABSTRACT

Luminescent detection of inorganic phosphate is carried out in an assay through the intermediary enzymatic production of ADP. ADP is converted to ATP which is used in a luminescent reaction. The assay can be used to monitor coupled enzyme reactions which use or generate inorganic phosphate and the modulation of such reactions.

### CROSS REFERENCE TO RELATED APPLICATIONS

This application claims priority to U.S. Provisional Application No. 61/777,002, filed Mar. 12, 2013, which is incorporated herein by reference in its entirety

### BACKGROUND

Accurate inorganic phosphate (Pi) detection typically encompasses the use of ³²P-radiolabelled substrates, but often such substrates are difficult to obtain. Radioactive methods also have limitations such as the need for highly skilled personnel and are not amenable to high-throughput screening. Although several non-radioactive methods have been developed, they suffer from extremely low sensitivity. For example, a widely-used malachite green-based inorganic phosphate detection system suffers from extremely low sensitivity.

Other methods for inorganic phosphate detection include fluorescent methods in which enzymes use inorganic phosphate to produce fluorescent products. For example, maltose phosphorylase can be used to convert maltose and inorganic phosphate to glucose and glucose 1-phosphate. The glucose produced may then be used by glucose oxidase to produce gluconolactone and H₂O₂. Using Horseradish Peroxidase as the catalyst, the H₂O₂ can be reacted with Amplex Red (non-fluorescent) to generate a highly fluorescent resorufin (Abs/Em: 563/587 nm). In another system, purine nucleoside phosphorylase is used to convert 2-amino-6-mercapto-7-methyl purine riboside (MESG) to ribose 1-phosphate and 2- amino-6-mercapto-7-methyl purine. The accompanying change in absorbance at 360 nm allows quantitation of inorganic phosphate consumed in the reaction. While these assay systems are improvements over the malachite green colorimetric assay, they have reduced sensitivity.

Other inorganic phosphate sensors use the E. coli phosphate-binding protein phoS. Using a thio-reactive environment sensitive fluorophore, binding of Pi to the phosphate-binding protein causes increased fluorescence. However, this method also suffers from low signal-to-background and signal-to-noise ratios.

### SUMMARY

In some embodiments, a method for detecting the presence or absence of or determining the amount of inorganic phosphate (Pi) in a solution is provided. The method includes contacting the solution with polynucleotide phosphorylase and poly adenylate (poly A) to convert the inorganic phosphate to ADP; converting the ADP produced from the inorganic phosphate to ATP with an enzyme that catalyzes the conversion of ADP to ATP; and detecting the ATP produced from the ADP using a luminescent reaction.

In some embodiments, a method for estimating the activity of an inorganic-phosphate generating enzyme is provided. Inorganic phosphate is generated in a solution with an inorganic-phosphate-generating enzyme, and the inorganic phosphate is detected by contacting the solution with polynucleotide phosphorylase to convert the inorganic phosphate to ADP. The ADP is converted to ATP with an enzyme that catalyzes the conversion of ADP to ATP, and the ATP produced from the ADP is detected using a luminescent reaction. The luminescent reaction provides a measure of the activity of the inorganic phosphate-generating enzyme. In some embodiments, the effect of a modulator, e.g., inhibitor or activator, on the activity of the inorganic-phosphate-generating enzyme is determined. In some embodiments, the inorganic-phosphate-generating enzyme is a pyrophosphatase, and the method provides a measure of the activity of the inorganic pyrophosphate-generating enzyme.

In some embodiments, kits are provided for detecting inorganic phosphate in a solution. The kits can include a polynucleotide phosphorylase, poly(A), an enzyme that catalyzes the conversion of ADP to ATP, a luciferase enzyme, and a substrate for the luciferase enzyme. The kits may optionally include one or more of a phosphate group donor, pyrophosphatase, one or more detergents, one or more buffer solutions and one or more salts.

Other aspects of the invention will become apparent by consideration of the detailed description and accompanying drawings.

### BRIEF DESCRIPTION OF THE DRAWINGS

FIG. 1: Schematic representation of the reactions of a bioluminescent assay for the detection of inorganic phosphate.

FIG. 2: Graphs showing detection of Pi using different concentrations of poly(A) in a bioluminescent reaction scheme.

FIG. 3: Graphs showing a comparison of a malachite green assay for Pi and the bioluminescent Pi assay of the present invention.

FIG. 4: Graph depicting a comparison of HPLC-grade and desalted poly(A) in a bioluminescent reaction scheme for Pi detection.

FIG. 5: Graph depicting the titration of hPNPase in a bioluminescent reaction scheme for Pi detection.

FIG. 6: Graph depicting the comparison of a one-step and two-step bioluminescent Pi-detection system.

FIG. 7. Graph showing suitable incubation times for a bioluminescent Pi-detection assay.

FIG. 8: Graphs depicting protein phosphatase activity measured using a bioluminescent Pi assay system (left graph, Yop: tyrosine phosphatase; center graph PTP 1B: tyrosine phosphatase; right graph PPase2a Serine/Threonine phosphatase).

FIG. 9: Graphs depicting lipid/inositol phosphatase activity measured using a bioluminescent Pi assay system.

FIG. 10: Graph depicting PTEN (phosphatase and tensin homolog) activity in immunoprecipitated samples from different cell types measured using a bioluminescent Pi assay system.

FIG. 11: Graph depicting 5′-nucleotidase activity using a bioluminescent Pi assay system.

FIG. 12: Graph depicting the detection of pyrophosphate using a bioluminescent Pi assay system.

FIG. 13: Graph depicting GAP dependent Ras GTPase activity using a bioluminescent Pi assay system.

### DETAILED DESCRIPTION

The invention provides compositions, kits and methods to determine or detect the presence or amount of inorganic phosphate (Pi) utilizing a luminescent detection system. The detection does not require an excitation light source as is needed for fluorescent systems, and thereby has a low background and high sensitivity. The high sensitivity permits the use of low amounts of enzymes and substrates for detection of inorganic phosphate.

The inorganic phosphate-detection assay suitably can be performed in two or more steps, which may occur consecutively or at least partially concurrently. In a first step, the appropriate substrate is contacted with assay components to initiate the reaction which forms ADP using Pi. The assay components may be provided separately, in any combination, or together as an assay reagent. The assay components suitably include buffer and components that convert the inorganic phosphate to ADP. In some embodiments, the assay components include a polynucleotide phosphorylase and a poly(A) substrate. In some embodiments, the assay components may also include one or more of enzymes, substrates and cofactors that form inorganic phosphate by one or more enzymatic reactions, or modulators (such as inhibitors or activators) of such enzyme reactions. The assay components may be substantially free of inorganic phosphates (Pi). In some embodiments, the assay components alone or in combination, contain less than about 10 nM, less than about 20 nM, less than about 50 nM, or less than about 100 nM inorganic phosphate.

In a second step, the ADP formed is contacted with a detection composition which converts the ADP to ATP, and then the ATP to light. In general, the methods to convert the ADP to ATP, and then the ATP to light include contacting the ADP formed in the first step with a composition comprising an ADP to ATP converting enzyme, such as a kinase, a luciferase, a luminogenic substrate, such as luciferin or a luciferin derivative that is a substrate of the luciferase, and optionally, if needed, a phosphate group-donor substrate for the ADP to ATP converting enzyme. In some embodiments, the detection components may be provided separately, in any combination or together as a single detection reagent. In some embodiments, the detection components and the assay components are provided as a single composition or in two or more separate compositions.

The luminescent reaction provides an indication of the presence or absence of or a measure of inorganic phosphate, e.g., amount, in a solution. When a measure of the inorganic phosphate in the solution is desired, the results from the luminescent reaction may be compared against one or more standards or control reactions. In some embodiments, the detection or measurement of inorganic phosphate is coupled to one or more enzyme reactions that directly or ultimately generate inorganic phosphate, and the luminescent reaction provides a measure, e.g., amount or activity, of the coupled enzyme(s), substrate(s), product(s), modulator(s) (such as inhibitors or activators), or combination thereof. In some embodiments, the assay can be used in a high-throughput assay format to screen for proteins, components or modulators of coupled enzyme reactions.

The methods, compositions and kits described herein can be used to detect or measure phosphate in in vitro biochemical assays. In some embodiments, biochemical characterization of Pi-generating enzymes may include measurement of IC₅₀, substrate specificity, the effect of inhibitors, activators, modulators, or any combination thereof In some embodiments, the enzymes are pure enzyme preparations.

The methods, compositions and kits described herein also can be used to detect or measure proteins which are obtained by immunoprecipitation from cell lysates which generate Pi, for example, in a cell-based assay. For example, phosphatase activities in cell lysates can be detected or measured. Cell lysates may be crude cell lysates, such as comprising soluble and insoluble cellular material, clarified cell lysates, such as by centrifugation, or purified cell lysates.

Many enzymatic reactions can be coupled to produce inorganic phosphate used in the methods kits and compositions described herein. The detection or measurement of inorganic phosphate facilitates detection or measurement of such enzymes, their substrates, products, modulators or any combination thereof Many enzymatic reactions which produce inorganic phosphate are therapeutically relevant drug targets. Exemplary enzymes that generate inorganic phosphate as a product of their enzymatic reaction include, without limitation, phosphatases (lipid and protein phosphatases), inositol phosphatases, transcarbamylase, phosphorylase, nucleotidases, ATPases, GTPases (e.g., small GTPases), and tubulin. Those of skill in the art would appreciate that other enzymes which produce Pi may be similarly coupled to the reactions described herein. The following enzymes, families of enzymes, substrates and products are only examples of the enzymes, enzyme families, substrates and products that can be assayed or screened using the methods, kits and compositions disclosed herein, and are not intended as limiting the scope of the invention.

A. Coupled Enzyme Reactions for Assaying or Screening

1. Phosphatases: EC. 3.1.3.XX, Phosphoric Monoester Hydrolases

a. Protein phosphatases: Protein phosphatases constitute a large family of enzymes. Posttranslational protein phosphorylation is important for signal transduction. Kinases add phosphate groups on proteins using primarily ATP as the phosphate donor. Phosphorylation is a reversible process and phosphorylated proteins can be dephosphorylated by phosphatases.

Abnormalities in protein phosphatases are associated with various pathologies including diabetes, obesity, cancer, neurodegenerative diseases and autoimmune diseases. Protein phosphatases are classified by their substrate specificity to Ser/Thr phosphatases (STP), protein-histidine phosphatases (PHP), protein Tyr phosphatases (PTP) and dual-specificity phosphatases (DSP) Inhibitors of protein phosphatases are sought as therapeutic strategies. Some inhibitors of proteins phosphatases that have entered clinical trials include PTP1B, SHP-2, CD45, and PP2A.

b. Lipid Phosphatases: Phosphorylation of lipids, including lipids with inositol rings, is important in signal transduction and in plasma membranes and vesicles. Lipid kinases convert the phospholipid PI3K PI(4,5)P2 to PIP3 a signaling second messenger. PIP3 levels in the cell are also regulated by the signaling proteins phosphatase and tensin homolog (PTEN) and phosphatidylinositol 3,4,5-trisphosphate 5-phosphatase (SHIP). Lipid phosphatases PTEN, SHIP, myotubularin, and sphingosine 1-phosphate phosphatase are involved in a number of diseases including cancer, inflammation, and diabetes. PTEN is one of the most frequently mutated genes in cancer.

Inositol phosphates are formed from PIP2 by the activity of PLC producing Ins(1,4,5)P3 and diacylglycerol. Inositol phosphates are phosphorylated and dephosphorylated by a series of kinase and phosphatases and are eventually used in regeneration of phospholipids. An example of such a phosphatase is Inositol-1′ monophosphatase (IMPase). Lithium, one of the commonly used drugs for bipolar disorders, targets IMPase. In certain embodiments, lipid phosphatases, including those described herein, can be assayed using water soluble lipids diC8 phosphoinositides (PIPs) in assays described herein.

c. Inositol Phosphatases/Sugar Phosphatases: Inositol phosphates include inositol 1,4,5-trisphosphate (Ins (1,4,5)P3 or IP₃), which is the source of various other inositol phosphates in the cell. IP₃ is produced by the activity of PI-PLC and can be converted to various kinds of inositol phosphates using various kinases and phosphatases. IP₃ is an important second messenger critical for calcium signaling. Phosphatidylinositol phosphatases like PTEN and SHIP also act on inositol phosphates. Various sugars are also phosphorylated by sugar kinases and dephosphorylated by sugar-specific phosphatases.

2. GTPases

GTPases are GTP-specific hydrolases; they convert GTP to GDP and Pi. Most GTPases have a very high affinity for both GTP and GDP. GTPases are active in their GTP bound state, and inactive when bound to GDP. By itself GTPases are very slow acting enzymes. To convert the GTPase from its activated GTP-bound form to GDP-bound form, GTPase activating proteins (GAPs) assist in hydrolyzing the bound GTP. The GDP formed in the process remains bound to the GTPase as it has high affinity for GDP (same as GTP), and a free-phosphate (Pi) is released. This class of enzymes includes heterotrimeric G-protein (Ga), small monomeric GTPases (Ras, Rho, Rac, cdc42, Rab, Ran, Arf, Rheb, Elongation Factor Tu (EF-Tu/EF-1A), protein-synthesizing GTPase, signal-recognition-particle, dynamin, and tubulin. Ras is the most commonly mutated protein in cancer. Small GTPases regulate cell proliferation, growth, cell division, migration, vesicular transport, nuclear transport, cytoskeletal processes. Heterotrimeric GTPases are associated with G-protein coupled receptors and are involved in GPCR-mediated signaling. Pi-released from GAP stimulated GTPase activity can be assayed using the assays described herein. GAPs are also signaling proteins. TSC2, the GAP for Rheb, when mutated causes various diseases like tuberous sclerosis and lymphangioleiomyomatosis. Cellular GAP, like TSC2, can be immunoprecipitated and assayed according to methods disclosed herein. For example, a GTP-loaded Rheb substrate can be included in the assay reagent to detect functional TSC2 GAP activity from cell lysates.

3. Enzyme coupled reactions to measure different products/enzyme function

a. Phospholipase C: PLCs are of two types PC-PLC (Phosphatidylcholine-specific-phospholipase C) and PI-PLC (Phosphatidylinositol-specific Phospholipase C). PI-PLCs are involved in cell signaling, regulation of phospholipid balance in the cell and also regulating calcium signaling. Using, for example, diC8 PIP as a substrate, Phospholipase C can convert PIP to inositol-1 phosphate and Diacylglycerol (DAG). The inositol-1 phosphate formed can be measured by using inositol-1 monophosphatases (IMPase) which are optionally included in the assay reagent. The inositol-1 phosphate is dephosphorylated forming myo-inositol and Pi. The Pi formed can then be utilized with the detection method of the present invention. The detection method is suitable for use with cell lysates, for example, by immunoprecipitating PLC from cell lysates and detecting PLC activity using the diC8 PIP as substrate and IMPase containing modified assay reagent. The assay suitably provides a Pi-PLC detection system that uses an antibody suitable for immunoprecipitation of PLC and does not require phosphospecific antibodies.

b. Sulfotransferase/Sialyltransferase: Sulfotransferases are a large group of enzymes that transfer sulfate from the donor substrate 3′ -phosphoadenosine-5′-phosphosulfate (PAPS) to various acceptor substrates, generating 3′-phosphoadenosine-5′-phosphate (PAP) as a by-product. The enzymes are involved, for example, in drug detoxification/xenobiotics and glycobiology. Traditional assays for these enzymes have involved use of radiolabeled substrates. By using 3′- or 5′- phosphatases in the method of the present invention, the amount of PAP formed can be indirectly measured, providing an indication of the activity of sulfotransferases. Golgi-resident PAP-specific 3′-phosphatase (gPAPP/ inositol monophosphatease IMPAD1), for example, can be used to release the 3′-phosphate from PAP, generating 5′-adenosine monophosphate (5′-AMP). In addition, CD73, a 5′-nucleotidase, can be used to release the 5′-phosphate. Using a similar principle, sialytransferases can also be analyzed using the methods kits and compositions disclosed herein. For sialyltransferases, CMP-sialic acid is a suitable donor. Upon activity of sialyltransferases, CMP is generated, which can be measured, for example, by including CD73 in the method of the present invention. CD73 is a 5′-nucleotidase, used to release the 5′-phosphate. The compositions, methods and kits described herein may be used to miniaturize sulfotransferase and sialyltransferase assays and provide assay formats amenable to high-throughput screening.

c. Nucleotidases: Nucleotidases are also phosphoric monoester hydrolase enzymes that fall in the same class as phosphatases. One such nucleotidase, 5′-nucleotidase (5′-NTase), is a clinically relevant biomarker and is elevated in serum levels children with liver and bone abnormalities. 5′-NTase converts nucleotide monophosphates (like AMP/GMP/CMP) to generate nucleosides (adenosine/guanosine/cytosine) and Pi. 5′-NTase can be used in a coupled biochemical assay with the methods described herein to measure activities of sulfotransferases and sialytransferases which are involved in drug metabolism and glycobiology.

d. PPi-detection: The detection of inorganic pyrophosphate (PPi) can be carried out and can be used to estimate of ATP-independent pyrophosphate generating enzyme activities and modulators. For example, soluble guanyl cyclase, HGPRT, PRPP-dependent phosphoribosyl transferase or nicotinamide phosphoribosyl transferase (NAMPT) are drug targets for various diseases. The pyrophosphate formed in these enzymatic reactions can be detected according to the methods, kits and compositions described herein by including pyrophosphatase in the methods, kits and compositions described herein.

Other Pi-generating enzymes, such as transcarbamylases and phosphorylases, can also be assayed using the methods, compositions and kits described herein.

The methods, compositions and kits described herein can be used to determine the effect of modulators on Pi-generating or consuming reactions described herein, which in turn allows the assessment of whether the modulator may function as a pharmaceutical drug. Modulators can be small molecules (including organic and inorganic molecules and synthetic and naturally occurring molecules). The methods may include controls in which samples are contacted with control substances whose effects on Pi-generating or consuming enzymes activity are known. Also, controls may include samples in which the Pi-generating enzyme, ADP to ATP converting enzyme or ATP-dependent luminescent enzyme and the test agent(s) are present together to assure that the modulator does not directly affect the Pi-generating or consuming enzyme, ADP to ATP converting enzyme and/or ATP-dependent luminescent enzyme activity.

B. Exemplary Components of the Methods, Kits and Compositions

The generation of ADP from Pi is suitably carried out using polynucleotide phosphorylase (PNPase). PNPase uses ribonucleotides (rNDPs) as the substrate for the polymerization reaction and can catalyze the 3′ to 5′ degradation of RNA polymers. Examples of PNPases include human PNPase, E. Coli PNPase, and Synechocystis PNPase, however, any suitable source of PNPase may be used in the methods, compositions and kits described herein, including synthetic, plant, bacterial and mammalian sources, so long as the PNPase has the capacity to consume Pi and generate ADP.

Suitable substrates for PNPase that may be used in the methods, kits and compositions include rNDP polymers such as poly(A). PNPase degrades poly(A) using Pi to generate ADP. Poly(A) can be suitably prepared using PNPase. Removal of contaminating ADP may be desirable prior to use of a PNPase-generated poly(A) in the assay. Poly(A) can also be chemically synthesized, for example, using high performance liquid chromatography (HPLC) using methods known in the art. The poly(A) may be suitably at least about a 5 mer, at least about a 10 mer, at least about a 15 mer or at least about a 20 mer and less than about a less than about 500 mer, less than about a 250 mer, less than about 100 mer, less than about a 75 mer, less than about a 50 mer, less than about a 40 mer, less than about a 30 mer or less than about a 25 mer. Other substrates for PNPase which generate ADP may also be used.

The ADP generated from the PNPase can be converted to ATP using an ATP-generating enzyme, such as a kinase, for example, adenylate kinase (myokinase), creatine kinase, or pyruvate kinase, with a phosphate group donor substrate. The ATP generated can then be consumed by an ATP-dependent enzyme, such as a luciferase, to generate light. FIG. 1 describes an exemplary reaction scheme for bioluminescent detection of inorganic phosphate as described herein.

Phosphate-group donors that can be used in the methods, compositions and kits include, for example, phosphocreatine, phosphoenolpyruvate or polyphosphate. The luminescent ATP-dependent enzyme for use in the methods, compositions and kits can include, for example, without limitation, beetle luciferase, e.g. Firefly luciferase, and the like. Any luciferase that retains the ability to generate luminescence when used in the assays described herein can be used. Optionally, the luciferases used in the compositions and methods disclosed herein have enhanced thermostability properties, chemostability properties, or a combination thereof.

The luminogenic substrate in the methods, compositions and kits can be, for example, a luciferin, a functional analog of luciferin, or a luciferin derivative. A luciferin derivative is a type of luminogenic molecule or compound having a substantial structure of D-luciferin and is a luciferase substrate. Examples include aminoluciferin, naphthyl and quinolyl derivatives, or luminogenic substrates disclosed in U.S. published application number 2007-0015790, the entire disclosure of which is incorporated by reference herein in its entirety.

The methods, compositions and kits described herein may also include a pyrophosphatase, such as inorganic pyrophosphatase. Pyrophosphatase facilitates the detection or measurement of enzyme reactions that produce pyrophosphate, by converting inorganic pyrophosphate (PPi) to inorganic phosphate (Pi).

The methods, compositions and kits disclosed herein that contain one or more enzymes may also comprise an enzyme stabilizing agent. The enzyme stabilizing agent can be any compound that stabilizes the enzyme, e.g., from degradation. Suitable enzyme stabilizing agents include proteins (such as bovine serum albumin, gelatin or PRIONEX® (purified gelatin)) or detergents (such as non-ionic detergents, e.g., THESIT® (hydroxypolyethoxydodecane)).

Components used in the methods, compositions or kits may be provided as one or more buffered solutions or in desiccated form with a buffer present that provides buffering upon reconstitution of the components with water or other solution. The buffer may provide a pH of about 6.0 to 8.0.

Kits can contain assay components which are provided in a single container, in separate containers or grouped together in appropriate combinations in separate containers. For example, the components for the generation of ADP can be packaged together in a container. The components for generating Pi, modulating Pi, or a combination thereof can be packaged together in a container, and components for the degradation of ATP in the luminescent reaction can be packaged together in a container. The components for the generation of ADP can be packaged together with the components for generating Pi, modulating Pi, or a combination thereof.

C. Methods and Assay Conditions

Methods disclosed herein facilitate detecting the presence or absence of inorganic phosphate (Pi) or determining the amount of inorganic phosphate (Pi) in a solution. The methods include contacting a solution suspected of containing Pi with a polynucleotide phosphorylase to convert the inorganic phosphate to ADP. The ADP produced from the inorganic phosphate is converted to ATP with an enzyme that catalyzes the conversion of ADP to ATP, and the ATP produced from the ADP is detected using a luminescent reaction. The solution can include a sample as described herein, such as cells, or a crude or purified cell extract.

The polynucleotide phosphorylase reaction may be proceed for a period of time of at least about 1 minute, at least about 2 minutes, at least about 5 minutes, at least about 10 minutes, at least about 15 minutes, at least about 20 minutes, or at least about 25 minutes, and less than about 24 hours, less than about 12 hours, less than about 5 hours, less than about 4 hours, less than about 3 hours, less than about 2 hours or less than about 1 hour.

The pH of the polynucleotide phosphorylase reaction may be at least about 5.5, at least about 5.6, at least about 5.7, at least about 5.8, at least about 5.9, at least about 6.0, at least about 6.1, at least about 6.2, at least about 6.3, at least about 6.4, at least about 6.5, at least about 6.6, at least about 6.7, at least about 6.8, at least about 6.9, at least about 7.0, at least about 7.1, at least about 7.2, at least about 7.3, at least about 7.4, and less than about 9.0, less than about 8.9, less than about 8.8, less than about 8.7, less than about 8.6, less than about 8.5, less than about 8.4, less than about 8.3, less than about 8.2, less than about 8.1, less than about 8.0, less than about 7.9, less than about 7.8, less than about 7.7 or less than about 7.6.

The reaction in which ADP is converted to ATP, for example with a kinase, may be allowed to proceed for a period of time of at least about 1 minute, at least about 5 minutes, at least about 10 minutes, at least about 15 minutes, at least about 20 minutes, at least about 25 minutes, at least about 30 minutes and less than about 24 hours, less than about 12 hours, less than about 6 hours, less than about 5 hours, less than about 4 hours, less than about 3 hours, less than about 2 hours, less than about 90 minutes, less than about 80 minutes, less than about 70 minutes, less than about 65 minutes, or less than about 60 minutes.

The pH of the reaction in which ADP is converted to ATP may be at least about 5.5, at least about 5.6, at least about 5.7, at least about 5.8, at least about 5.9, at least about 6.0, at least about 6.1, at least about 6.2, at least about 6.3, at least about 6.4, at least about 6.5, at least about 6.6, at least about 6.7, at least about 6.8, at least about 6.9, at least about 7.0, at least about 7.1, at least about 7.2, at least about 7.3, at least about 7.4, and less than about 9.0, less than about 8.9, less than about 8.8, less than about 8.7, less than about 8.6, less than about 8.5, less than about 8.4, less than about 8.3, less than about 8.2, less than about 8.1, less than about 8.0, less than about 7.9, less than about 7.8, less than about 7.7 or less than about 7.6.

The luminogenic reaction which consumes ATP may proceed for a period of time of at least about 1 minute, at least about 5 minutes, at least about 10 minutes, at least about 15 minutes, at least about 20 minutes, at least about 25 minutes, at least about 30 minutes and less than about 24 hours, less than about 12 hours, less than about 6 hours, less than about 5 hours, less than about 4 hours, less than about 3 hours, less than about 2 hours, less than about 90 minutes, less than about 80 minutes, less than about 70 minutes, less than about 65 minutes, or less than about 60 minutes.

The pH of the luminogenic reaction may be at least about 5.5, at least about 5.6, at least about 5.7, at least about 5.8, at least about 5.9, at least about 6.0, at least about 6.1, at least about 6.2, at least about 6.3, at least about 6.4, at least about 6.5, at least about 6.6, at least about 6.7, at least about 6.8, at least about 6.9, at least about 7.0, at least about 7.1, at least about 7.2, at least about 7.3, at least about 7.4, and less than about 9.0, less than about 8.9, less than about 8.8, less than about 8.7, less than about 8.6, less than about 8.5, less than about 8.4, less than about 8.3, less than about 8.2, less than about 8.1, less than about 8.0, less than about 7.9, less than about 7.8, less than about 7.7 or less than about 7.6.

Light generated in the luminescent reaction is typically detected with a luminometer although other detection means may be used. The presence of light greater than background level indicates the presence of ATP in the sample. The background level of luminescence is typically measured in the same matrix, but in the absence of the sample. Suitable control reactions are readily designed by one of skill in the art. Luciferases may allow for multiple analyses of a sample over time or analysis of many samples over time. Light can be detected qualitatively or quantitatively.

Quantifying the amount of emitted light quantifies the amount of ATP, and thus the amount of ADP produced by the PNPase in the assay from Pi. Thus, quantitation of ATP allows for quantitation of PNPase activity, the effect of a modulator on PNPase activity, the amount of Pi, the activity of a coupled Pi-generating or Pi-consuming enzyme or the effect of a modulator of a coupled Pi-generating or Pi-consuming enzyme reaction.

Quantitative ATP values are realized, for example, when the quantity of light emitted from a test sample in the assay is compared to the quantity of light emitted from a control sample or to a standard curve determined by using known amounts of ATP, and the same or substantially similar luciferase and reaction conditions (i.e., temperature, pH, etc.). It is understood that quantification involves subtraction of background values. Qualitative ATP values are realized when the luminescence emitted from one sample is compared to the luminescence emitted from another sample without a need to know the absolute amount of ATP converted from ADP present in the samples, e.g., a comparison of samples in the presence or absence of a test agent. Many such experiments can readily be designed by one of ordinary skill in the art.

The methods can be used to detect or determine the activity of enzymes which generate inorganic phosphate such that the luminescent reaction provides a measure of the activity of the inorganic phosphate-generating enzyme. The methods can also be used to detect or determine the activity of enzymes which generate inorganic pyrophosphate such that the luminescent reaction provides a measure of the activity of the inorganic pyrophosphate-generating enzyme. In this case, pyrophosphatase is included in the reaction mixture that converts PPi to Pi.

The reactions that generate the inorganic phosphate, the polynucleotide phosphorylase reaction, the reaction in which ADP is converted to ATP and the luminogenic reaction which consumes ATP may be performed sequentially or at least partially concurrently, such as in a homogeneous assay. For example, the reaction that generates inorganic phosphate can occur concurrently with the conversion of inorganic phosphate to ADP; the conversion of ADP to ATP can occur concurrently with the conversion of inorganic phosphate to ADP; the luminescent reaction can occur concurrently with the conversion of ADP to ATP; or any combination thereof. Each of the reactions may also be conveniently carried out in a single reaction vessel.

D. Samples for use with the Methods, Kits and Compositions

A sample for use in the methods, kits or compositions described herein may comprise cells, a cell lysate, a subcellular fraction of a lysate, such as a membrane fraction or a cellular sample, and includes physiological samples. Cell lysates may be crude cell lysates, such as lysates comprising soluble and insoluble cellular material, clarified cell lysates, such as by centrifugation, or purified cell lysates. A sample for using in the methods, kits or compositions described herein may comprise a Pi-generating enzyme(s) that is a pure enzyme preparation.

Cells that may be used with the methods compositions and kits described herein include prokaryotic and eukaryotic cells, including plant cells and vertebrate cells, for instance, mammalian cells including, but not limited to, human, non-primate human, bovine, equine, ovine, swine, caprine, feline, canine, mink, rodent or avian cells. A sample comprising cells may be treated so as to permeabilize or lyse the cells in the sample. Methods for permeabilization, lysis or disruption of cells or subcellular fractions thereof are well known in the art. A wide variety of equipment is available for mechanical disruption including sonicators (ultrasonic generators), a dounce, mortar and pestle, or French presses. Cells can be disrupted (yielding cell lysates) by osmotic shock, by treatments such as a series of freeze-thaw cycles or a rapid alteration of the ionic strength of the environment, or by the use of agents that directly disrupt cell membranes such as enzymes like lysozyme or chemical agents such as detergents or surfactants, such as zwitterionic and nonionic detergents, or cationic detergents DTAB or CTAB, and antibacterial agents such as polymyxin B and chlorhexidine.

The cells in a sample, e.g., a sample which includes eukaryotic cells such as yeast, avian, plant, insect or mammalian cells, including but not limited to human, simian, murine, canine, bovine, equine, feline, ovine, caprine or swine cells, prokaryotic cells, cells from two or more different organisms, or cell lysates, may not have been genetically modified via recombinant techniques (non-recombinant cells), or may be recombinant cells which are transiently transfected with recombinant DNA and/or the genome of which is stably augmented with a recombinant DNA, or which genome has been modified to disrupt a gene, e.g., disrupt a promoter, intron or open reading frame, or replace one DNA fragment with another. The recombinant DNA, or replacement DNA fragment, may encode a Pi-generating enzyme to be detected by the methods described herein, a moiety which alters the level or activity of the Pi-generating enzyme to be detected and/or a gene product unrelated to the molecule or moiety that alters the level or activity of the Pi-generating enzyme.

It will be apparent to those of skill in the art that variations may be applied to the compositions and methods described herein and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

It is to be understood that the invention is not limited in its application to the details of construction and the arrangement of components set forth in the following description. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The following examples are for purely illustrative purposes and are not to be construed as limiting the scope of the invention.

### Example 1

### Bioluminescent Detection of Pi using Different Poly(A) Concentrations

Chemically synthesized HPLC grade poly(A) (20 mer) was tested in the reaction scheme according to FIG. 1. A phosphate standard was serial diluted from 100-0 μM in buffer containing 40 mM Tris-HCl, pH 7.5, 20 mM MgCl₂ in 5 μl. To this, 5 μl Pi-assay reagent (40 mM Tris-HCl, pH7.5, 10 mM MgCl2, 0.4 U/ml hPNPase, 1 μM, 2 μM or 5 μM poly(A)) was added containing different amounts of poly(A). The 10 μl reaction was then incubated for 1 hour at room temperature. To this, 10 μl of luciferase detection reagent (ADP-Glo® Kinase assay (ADP-Glo® Detection reagent) commercially available from Promega) was added. The reaction was incubated for 30 minutes and luminescence detected. The results are shown in FIG. 2.

Under these assay conditions, when a lower poly(A) concentration was used, the assay was more sensitive and could detect lower concentrations of Pi. Increasing poly(A) concentration increased the background progressively. Using 1 μM poly(A) for the assay, the linear range was in the lower Pi concentrations and plateaued beyond 12.5 μM. When using 5 μM poly(A), the sensitivity was lower in the low Pi concentrations, but the assay maintained linearity over a greater Pi range (up to the last tested concentration of 100 μM). The use of 1 μM poly(A) provided an accurate measurement of 0.1-0.2 μM Pi (1-2 picomoles).

### Example 2

### Sensitivity of the Bioluminescent Detection Assay

Inorganic phosphate (KHPO₄) was serial diluted from 100-0 μM and detected with either a standard malachite green detection assay or with a bioluminescent detection system utilizing poly(A), human polynucleotide phosphorylase, phosphoenolpyruvate, pyruvate kinase, luciferase and luciferin. As shown in FIG. 3, the malachite green assay could detect Pi above 10 μM whereas the bioluminescent system could detect as little as 0.1 μM Pi.

### Example 3

### Bioluminescent Detection of Pi Using Poly(A) from Different Sources in a PP2A Phosphatase Assay

HPLC-grade or desalted-grade poly(A), both as 20 mers, were each used in a bioluminescent detection system utilizing human polynucleotide phosphorylase, phosphoenolpyruvate, pyruvate kinase, luciferase and luciferin to measure PP2A phosphatase. FIG. 4 shows a comparison of the use of HPLC-grade and desalted-grade poly(A) in a PP2A phosphatase assay using phosphorylated peptide as a substrate. Both desalted-grade and HPLC-grade poly(A) facilitate the detection of Pi-released by the phosphatase. HPLC-grade poly(A) gave a higher signal-to-background ratio.

### Example 4

### Titration of Human Polynucleotide Phosphorylase

The amount of hPNPase for performing Pi-detection was determined Human PNPase (stock concentration 10 U/ml) was serial diluted from 1-0 U/ml in 40 mM Tris-HCl, pH 7.5 and 20 mM MgCl₂. To 10 μM Pi, 2 μM poly(A) was added. The reaction was incubated for 1 hour producing ADP. The ADP formed was detected using a composition comprising luciferase, luciferin, pyruvate kinase and phosphoenolpyruvate. A concentration of 0.2 U/ml hPNPase provided about 100-fold increase in luminescence (FIG. 5).

### Example 5

### One Step and Two Step Detection of Pi Produced by a Phosphatase

The efficacy of the Pi detection assay of the present invention was assessed as a one-step process, where the Pi-generating enzymatic reaction (PTP1B-Tyr phosphatase using a phosphorylated peptide substrate) was coupled to hPNPase mediated poly(A) degradation and ADP generation in a single step. The efficacy was also assessed as a two-step process where the phosphatase reaction was stopped using universal phosphatase inhibitor sodium orthovanadate (Na₂VO₃), and then ADP from the phosphate was generated using hPNPase mediated poly(A) degradation. Both one and two step processes facilitated detection of Pi and the PTP1B_Tyr-phosphatase with increased sensitivity using the one step process (FIG. 6).

### Example 6

### Incubation Times for Pi Assay

A variety of incubation times for performing the Pi- and ADP-generating step were tested. The enzymatic Pi-generation reaction was coupled to generation of ADP, and PTP1B-Tyr phosphatase was used to generate Pi. PTP1B-Tyr phosphatase was titrated from 20-0 μg/ml to which the assay reagent (0.4 U/ml hPNPase, 1-2 μM poly(A) in 50 mM Tris-HCl, pH 7.5, 10 mM MgCl₂ and 1 mM DTT) containing 40 μM phosphor-Tyr peptide substrate was added. The reactions were incubated for 15, 30, 60 or 120 minutes, detection reagent was added, and incubated for 30 minutes. Higher luminescence was observed with longer incubation times (FIG. 7).

### Example 7

### Protein Phosphatase Activity Measured Using the Bioluminescent Pi Assay System

Two protein-Tyr phosphatases (PTPs), Yop and PTP1B, and one Ser/Thr phosphatase, PP2A, were tested in the Pi assay of the present invention. Phosphatases were serial diluted in 50 mM Tris-HCl, pH 7.5, 10 mM MgCl₂ and 1 mM DTT, and phospho-protein substrates were added to the assay reagent (0.4 U/ml hPNPase, 1-2 μM poly(A) in 50 mM Tris-HCl, pH 7.5, 10 mM MgCl₂ and 1 mM DTT) at a concentration of 40 μM. The reaction proceeded for 1 hour in a 10 μl reaction in wells of a 384-low volume plate. 10 μl of detection reagent comprising pyruvate kinase, phosphoenolpyruvate, luciferin and luciferase was then added. Luminescence was detected after 30 minutes. FIG. 8 shows that the assay is sensitive in detecting protein phosphatase activity using the Pi assay of the present invention.

### Example 8

### Lipid/Inositol Phosphatase Activity Measured Using the Bioluminescent Pi Assay System

PTEN and SHIP2, both of which can use Ins(1,3,4,5)P4 and PtdIns(3,4,5)P3 as a substrate, were assayed. Water soluble PIP3 derivatives diC8 PIP3 (commercially available from Echelon Bioscience) were used as alternatives to water-insoluble PIP3. PTEN and SHIP2 were serial diluted in 50 mM Tris-HCl, pH 7.5, 10 mM MgCl₂ and 1 mM DTT. Then, diC8 PIP3 or IP4 were added to the assay reagent (0.4 U/ml hPNPase, 1-2 μM poly(A) in 50 mM Tris-HCl, pH 7.5, 10 mM MgCl2, 1 mM DTT) at a concentration of 40 μM. The reaction was allowed to proceed for 1 hour in a 10 μl reaction in wells of a 384-low volume plate. 10 μl of detection reagent was then added, and luminescence was detected after 30 minutes. FIG. 9 shows that PTEN accepts PIP3 as a more favorable substrate compared to IP4 while SHIP2 has no preference for either IP4 or PIP3.

### Example 9

### Cell-based Phosphatase Activity Assay

The PTEN-deficient cell line, Jurkat T cells, was compared with PTEN cell lines, A431 and HEK293). Cells were cultured in appropriate culture media, and 2×10⁶ cells lysed in 1 ml lysis buffer containing 50 mM Tris pH 8.0, 150 mM NaCl, 1 mM EDTA, 5% Glycerol and 1% Triton X-100 containing protease inhibitors. Cell lysates were centrifuged to remove the insoluble pellet and then incubated with 10 μl PTEN antibody (commercially available from Cell Signaling Technology) for 3 hrs. To this mixture, 100 μl of Protein A/G slurry was added and incubated overnight. The beads were then washed three times in PTEN reaction buffer (50 mM Tris HCl, pH 7.4, 150 mM NaCl, 2.7 mM KCl, 10 mM MgCl₂ and 5 mM DTT). After washing, 40 μl of assay reagent containing 40 μM diC3 PIP3 was added to the beads and incubated for 3 hours.

Immunoprecipitation reactions were performed on each sample. Briefly, the beads were spun down, and the supernatant collected. 10 μl of supernatant was dispensed into wells of a 384-well plate. 10 μl of detection reagent was added, incubated for 30 minutes, and luminescence was detected. FIG. 10 shows that Jurkat T cells lacking functional PTEN show minimal activity when assayed with the assay reagent described herein, whereas the A431 and HEK293 cells, which have functional PTEN, show much higher PTEN activity.

### Example 10

### Nucleotidase

5′-NTase was serial diluted in 50 mM Tris-HCl, pH 7.5, 10 mM MgCl₂ and 1 mM DTT. AMP (substrate for 5′-NTase) was added to the assay reagent (0.4 U/ml hPNPase, 1-2 μM poly(A) in 50 mM Tris-HCl, pH 7.5, 10 mM MgCl₂ and 1 mM DTT) at a concentration of 40 μM. The reaction was incubated for 1 hour in a 10 μl reaction in wells of a 384-low volume plate. 10 μl of Pi detection reagent was added, and luminescence detected after 30 minutes. FIG. 11 shows that the Pi assay system is highly sensitive in detecting 5′-NTase activity. It is expected that detection of sulfotransferases/sialyltransferases can be achieved using a coupled enzymatic reaction using 5′-NTase according to these methods of the present invention.

### Example 11

### PPi-detection

ATP-independent PPi producing enzymes.

PPi was serial diluted in 50 mM Tris-HCl, pH 7.5, 10 mM MgCl₂ and 1 mM DTT. Pyrophosphatase (2 U/ml) was added to the assay reagent. 10 μl of Pi detection reagent was added, and luminescence detected after 30 minutes. FIG. 12 shows that the Pi assay system is sensitive in detecting pyrophosphate (PPi).

### Example 13

### GTPase Detection

To test the feasibility of assaying GTPase activity using the Pi detection system of the present invention, Ras GTPase was combined with GTP and incubated in the presence or absence of Ras GAP NF1 (neurofibromatosis). Ras GTPase is a very slow acting GTPase, but, in the presence of GAP, the GTPase activity is increased several fold. In the presence of NF1, the GTPase activity was increased four-fold over the intrinsic GTPase activity (FIG. 13).

1. A method for detecting the presence or absence of or determining the amount of inorganic phosphate (Pi) in a solution, the method comprising:
(a) contacting the solution with polynucleotide phosphorylase to convert the inorganic phosphate to ADP;
(b) converting the ADP produced from the inorganic phosphate to ATP with an enzyme that catalyzes the conversion of ADP to ATP; and
(c) detecting the ATP produced from the ADP using a luminescent reaction.

2. The method of claim 1, wherein the conversion of ADP to ATP occurs concurrently with the conversion of inorganic phosphate to ADP.

3. The method of claim 1, wherein the luminescent reaction occurs concurrently with the conversion of ADP to ATP.

4. The method of claim 1, wherein steps (a) (b) and (c) are carried out as a homogeneous assay.

5. The method of claim 1, wherein steps (a) (b) and (c) are performed in a single reaction vessel.

6. The method of claim 1, wherein the solution comprises a crude cell lysate.

7. The method of claim 1, further comprising generating inorganic phosphate in the solution with an inorganic phosphate-generating enzyme, and wherein the luminescent reaction provides a measure of the activity of the inorganic phosphate-generating enzyme.

8. The method of claim 7, further comprising modulating the activity of the inorganic phosphate-generating enzyme with a composition comprising a modulator, wherein the luminescent reaction provides a measure of the effect of the modulator on the activity of the inorganic phosphate-generating enzyme.

9. The method of claim 7, wherein the measure is quantitative.

10. The method of claim 7, wherein the generation of inorganic phosphate occurs concurrently with the conversion of inorganic phosphate to ADP.

11. The method of claim 7, wherein the generation of inorganic phosphate occurs prior to the conversion of inorganic phosphate to ADP.

12. The method of claim 7, wherein the inorganic phosphate-generating enzyme is a lipid phosphatase, protein phosphatase, inositol phosphatase, transcarbamylase, phosphorylase, nucleotidase, ATPase, GTPase or tubulin.

13. The method of claim 7, wherein the inorganic phosphate-generating enzyme is a pyrophosphatase and further comprising generating inorganic pyrophosphate in the solution with an inorganic-pyrophosphate-generating enzyme, and wherein the luminescent reaction provides a measure of the activity of the inorganic pyrophosphate-generating enzyme.

14. The method of claim 1 wherein the enzyme that catalyzes the conversion of the ADP to ATP is a pyruvate kinase.

15. The method of claim 1 wherein the luminescent reaction comprises a luciferase enzyme and a substrate for the luciferase enzyme.

16. The method of claim 15, wherein the luciferase enzyme is a recombinant luciferase enzyme.

17. The method of claim 15, wherein the luciferase enzyme is a thermostable luciferase enzyme, chemostable luciferase enzyme, or a combination thereof.

18. A kit for detecting inorganic phosphate in a solution, the kit comprising:
(i) a polynucleotide phosphorylase;
(ii) poly(A)
(iii) an enzyme that catalyzes the conversion of ADP to ATP;
(iv) a luciferase enzyme; and
(v) a substrate for the luciferase enzyme.

19. The kit of claim 18 further comprising one or more of:
(vi) a phosphate group donor;
(vii) one or more detergents;
(viii) one or more buffer solutions; and/or
(ix) one or more salts.

20. The kit of claim 19, comprising a phosphate group donor, wherein the enzyme that catalyzes the conversion of the ADP to ATP is a pyruvate kinase, and the phosphate group donor is phosphoenolpyruvate.

21. The kit of claim 18, wherein a first composition comprises (i) and (ii) and a different composition comprises (iii), (iv) and (v).

22. The kit of claim 18, wherein the kit further comprises pyrophosphatase.
